(19) United States
(12) Patent Application Publication
(10) Pub. No.: US 2024/0209140 A1
(43) Pub. Date: Jun. 27, 2024

(54) HYDROXYL-FUNCTIONAL THIOETHER COMPOUNDS AND THEIR USE IN CURABLE COMPOSITIONS

(71) Applicant: BASF Coatings GmbH, Muenster (DE)

(72) Inventors: Christian BECKHAUS, Muenster (DE); Günter KLEIN, Muenster (DE); Vincent COOK, Muenster (DE); Ulrike WENKING, Muenster (DE)

(21) Appl. No.: 18/553,068

(22) PCT Filed: Apr. 28, 2022
(86) PCT No.: PCT/EP22/61299

§ 371 Date: Sep. 28, 2023

(30) Foreign Application Priority Data
Apr. 28, 2021 (EP) 21170891.2

(51) Int. Cl. C08G 18/38; B05D 7/00

(52) U.S. Cl. CPC C08G 18/3876; B05D 7/536; B05D 2503/00; C08G 2150/00; C08G 2170/00; C08G 2190/00

(57) ABSTRACT

Disclosed herein is a hydroxyl-functional thioether compound having formula (I)

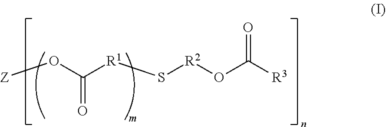

where Z is an n-valent hydrocarbyl group, optionally containing one or more moieties selected from the group consisting of an ether moiety, a thioether moiety and an isocyanurate moiety; R¹ is a linear or branched alkylene group; R² is selected from the group consisting of CH₂CH(OH)CH₂ and CH(CH₂OH)CH₂; R³ is a linear or branched alkyl group; m=0 or 1; and n=2 to 10. Further disclosed herein are a method of producing the same, a thermally curable composition containing the same and a method of using the hydroxyl-functional thioether compounds as reactive diluents in the thermally curable composition. Additionally disclosed herein are multilayer coatings where at least one layer is formed from such a composition, multilayer coated substrates and a method of producing such multilayer coatings.

The present invention relates to hydroxyl-functional compounds, their manufacture and use in thermally curable compositions as well as such compositions. The present invention further relates to multilayer coatings, wherein at least one layer is formed from such composition, a thus coated substrate and a method for producing such multilayer coating.

### Technological Background

In general, commercial coating compositions can be subsumed under physically drying and chemically crosslinking coating compositions. Amongst the chemically crosslinking coating composition it is distinguished between radiation curable coating compositions, where crosslinking is initiated by means of radiation, such as UV radiation and thermally curable coating compositions, where crosslinking is typically achieved by self-crosslinking of preferably polymeric species or crosslinking of polymers carrying functional groups with so-called crosslinking agents carrying functional groups, which react with the functional groups of the afore-mentioned polymers, thus forming a cured, i.e., crosslinked coating.

The present invention belongs to the field of thermally crosslinking coating compositions of the latter type. They can be formulated as one-pack or as two-pack compositions, in both of which essentially polymeric species are crosslinked with crosslinking agents.

In such prior art compositions, the polymeric binders often carry hydroxyl groups, while the crosslinking agents belong to the group of free or blocked diisocyanates and/or polyisocyanates or to the group of aminoplast resins such as melamine-formaldehyde resins.

In many cases the polymeric main binder to be crosslinked with the crosslinking agent essentially determines the viscosity of the coating composition and thus limits the possible application methods. To overcome this problem, it is common to utilize low molecular weight species, which are apt to reduce the viscosity compared to systems wherein only higher molecular weight polymeric binders are employed. Since such low molecular weight species need to be incorporated into the crosslinking network, they typically carry groups which are also reactive towards the reactive groups comprised in the crosslinking agent. Due to their viscosity reducing effect, i.e., diluting effect and their reactivity, such low molecular weight species are also called reactive diluents.

Besides their use in reducing the viscosity of crosslinkable compositions, they may also be employed to increase the solids content of compositions, thus replacing organic volatile ingredients such as organic solvents. Consequently, such compositions are more environmentally friendly than conventional compositions, which typically comprise a significantly higher amount of volatile organic compounds, so-called VOC.

WO 2013/072481 A1 discloses reactive diluents obtainable as reaction products of a polyol, namely glycerol with a carboxylic acid glycidyl ester. The thus obtained reactive diluents comprise hydroxyl groups which are reactive towards crosslinking agents from the group of polyisocyanates, aminoplast resins or triazines such as TACT. Such combination of reactive diluent and crosslinking agent can be employed in compositions which are suitable as coating compositions, adhesives and sealants, particularly those which further contain hydroxy functional polymers. The coating compositions disclosed in WO 2013/072481 A1 have a good levelling behavior.

It is also known to use thiol-functional reactive diluents in curable two-pack compositions along with hydroxyl-functional binders, or alone, since thiol groups as well as hydroxyl groups are known to react with crosslinking agents carrying isocyanate groups. In such cases polymeric networks are built, which contain thiourethane groups. The use of compounds containing two or more thiol groups in such reactions along with hydroxyl-functional binders typically accelerates the reaction rate, but also reduces the bubble formation, even in thick layers, thus leading to improved cured products. Such low molecular weight polythiols are e.g., available from Bruno Bock Chemische Fabrik GmbH & Co. KG, Marschacht, Germany under the tradename Thiocure®.

In yet another prior art approach the afore-mentioned low molecular weight polythiols are directly reacted with polyepoxides, thus producing crosslinked networks containing thioether links and hydroxyl groups.

However, in both of the afore-mentioned application systems, compounds having free thiol groups are used in the crosslinking reaction. Such components generally possess an unpleasant odor and are to be avoided in the target systems.

It was an aim of the present invention to provide a new class of reactive diluents. However, it was a further aim of the present invention to provide crosslinkable compositions which produce cured networks having an excellent chemical and scratch resistance. Such compositions should further show a good balance between levelling properties and sag resistance, preferably at higher film thicknesses, which often are competing properties.

### SUMMARY

The afore-mentioned aims were achieved by providing a hydroxyl-functional thioether compound having formula (I)

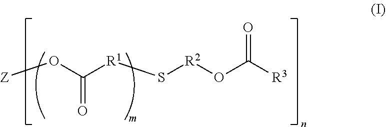

wherein
Z is an n-valent hydrocarbyl group, optionally containing one or more moieties selected from an ether moiety, a thioether moiety and an isocyanurate moiety;
R¹ is a linear or branched alkylene group;
R² is selected from CH₂CH(OH)CH₂ and CH(CH₂OH)CH₂;
R³ is a linear or branched alkyl group;
m=0 or 1; and
n=2 to 10.

The afore-mentioned hydroxyl-functional thioether compound of formula (I) is herein also denoted as “hydroxyl-functional thioether compound of the invention” or “reactive diluent of the invention”.

Further object of the present invention is a method of producing the hydroxyl-functional thioether compound of the invention, wherein one or more species Z—[O(C═O)R¹)ₘ—SH]ₙ are reacted with one or more species of formula (II)

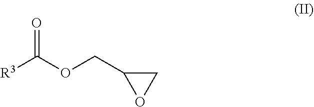

and
that in case of m=1 the one or more species Z—[O(C═O)R)ₘ—SH]ₙ are obtainable by reacting one or more species Z[OH]ₙ with n species of HOOC—R¹—SH; wherein Z, R¹, R³, m and n are defined as for the hydroxyl-functional thioether compound of the invention.

The afore-mentioned method of producing the hydroxyl-functional thioether compound of the invention is herein also denoted as “production method of the invention”.

Another object of the present invention is a thermally curable composition, comprising
(A) one or more hydroxyl-functional thioether compounds of the invention; and
(B) one or more crosslinking agents, which are reactive with the hydroxyl groups of the one or more hydroxyl-functional thioethers (A).

The afore-mentioned thermally curable composition is herein also denoted as “thermally curable composition of the invention” or just “curable composition of the invention”.

Yet another object of the present invention is the use of the hydroxyl-functional thioether of the invention as a reactive diluent in thermally curable compositions.

The afore-mentioned use is herein also denoted as “use according to the invention”.

The present invention further provides a multilayer coating comprising at least two coating layers wherein at least one of the layers is formed from a thermally curable composition and a thus coated substrate, herein denoted as “multilayer coating of the invention” and “multilayer coated substrate according to the invention”.

A further object of the present invention is a method of producing the multilayer coating of the present invention on a substrate, the method comprising the steps of:
(i) applying at least one basecoat composition on a substrate to form a basecoat layer and subsequently
(ii) applying at least one topcoat composition, onto the basecoat composition to form a topcoat layer, followed by
(iii) curing of the fully cured coating layers at a temperature in the range of 20° C. to 200° C.,
   wherein at least one basecoat composition or at least one topcoat composition is a thermally curable composition according to the invention.

### DETAILED DESCRIPTION

In the following, the invention is described in more detail by referring to preferred features and embodiments.

### Hydroxyl-Functional Thioether Compounds of Formula (I)

Preferably, in the hydroxyl-functional thioether compound of formula (I),

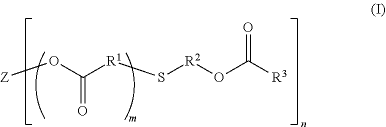

Z is an n-valent hydrocarbyl group containing 2 to 16 carbon atoms, optionally containing one or more ether moieties and/or one or more thioether moieties; and/or
R¹ is a linear or branched alkylene group containing 1 to 6 carbon atoms; and/or
R² is selected from CH₂CH(OH)CH₂ and CH(CH₂OH)CH₂; and/or
R³ is a linear or branched alkyl group 1 to 16 carbon atoms; and/or
m=0 or 1; and/or n=2 to 8.

It is to be emphasized that the preferred limitations in the preceding paragraph, which are separated by “and/or” are independent from each other and each one of the limitations can independently be combined with another or the broader definition of formula (I) found under the headline “SUMMARY”, above. However, it is particularly preferred that all afore-mentioned limitations are combined in one embodiment of the hydroxyl-functional thioether compounds of the invention, i.e., the “and/or” is replaced by “and”.

Even more preferred, the n-valent hydrocarbyl group Z contains 2 to 10 carbon atoms; and/or the linear or branched alkylene group R¹ contains 1 to 3 carbon atoms, such as 1, 2 or 3 carbon atoms; and/or the linear or branched alkyl group R³ contains 1 to 10, preferably 3 to 9 carbon atoms; and/or n=2 to 6. Again, these limitations, which are separated by “and/or” are independent from each other and each one of the limitations can independently be combined with another or with any of the afore-mentioned embodiments. However, it is particularly preferred that all limitations in this paragraph are combined in one embodiment of the hydroxyl-functional thioether compounds of the invention, i.e., the “and/or” is replaced by “and”.

In any of the hydroxyl-functional thioether compound according to the invention, R² is preferably CH₂CH(OH)CH₂.

If m=1 in the above formula (I), Z is preferably an n-valent hydrocarbyl group or an n-valent hydrocarbyl group containing one or more ether moieties; while, if m=0 in the above formula (I), Z is preferably an n-valent hydrocarbyl group containing one or more thioether moieties.

In all embodiments of the present invention the n-valent hydrocarbyl groups Z are preferably aliphatic hydrocarbyl groups encompassing cycloaliphatic hydrocarbyl groups, most preferred saturated aliphatic hydrocarbyl groups. Preferred examples for n-valent hydrocarbyl groups Z are as follows. The afore-mentioned hydrocarbyl groups may optionally contain one or more moieties selected from an ether moiety, a thioether moiety and an isocyanurate moiety.

For n=2, group Z is preferably a linear or branched alkylene group having 2 to 10, more preferred 2 to 8 and most preferred 2 to 6, such as 2, 3 or 4 carbon atoms; or a linear or branched alkylene ether group having 4 to 10, more preferred 4 to 8 and most preferred 4 to 6 carbon atoms.

For n=3, group Z is preferably selected from
H₂C—CH—CH₂,

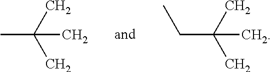

For n=4, group Z is preferably selected from

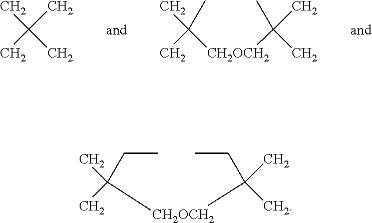

For n=6, group Z is preferably

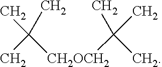

Most preferred, the group R³—C(═O)O in formula (I) is a trialkyl acetate group, even more preferred a so-called neodecanoyl group, derived from neodecanoic acid. Herein, neodecanoic acid denotes a carboxylic acid or mixture of carboxylic acids with the common structural formula C₁₀H₂₀O₂, a molecular weight of 172.26 g/mol, and the CAS number 26896-20-8. The common property of the neodecanoic acids is, that they can be subsumed under the general term “trialkyl acetic acid” having three alkyl groups at the carbon which is bound to the COOH group. Neodecanoic acid(s) typically include 2,2,3,5-tetramethylhexanoic acid, 2,4-dimethyl-2-isopropylpentanoic acid, 2,5-dimethyl-2-ethylhexanoic acid, 2,2-dimethyloctanoic acid and 2,2-diethylhexanoic acid.

### Method for Producing the Hydroxyl-Functional Thioether Compounds of Formula (I)

The hydroxyl-functional thioether compounds as defined above can be produced by reacting
(a) one or more species Z—[O(C═O)R¹)ₘ—SH]ₙ with
(b) one or more species of formula (II)

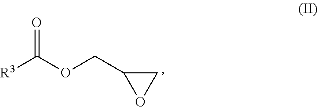

wherein Z, R¹, R³, m and n being defined as for the hydroxyl-functional thioether compounds of the invention.

In this reaction, the SH groups cause a ring-opening of the oxirane ring present in formula (II), thus forming residue R². In most cases ring-opening will lead to a residue R² being CH₂CH(OH)CH₂, thus containing a secondary OH group. However, in some cases ring-opening leads to the formation of residue CH(CH₂OH)CH₂, containing a primary OH group, wherein typically the “CH” moiety is bound to the adjacent S atom and the terminal CH₂ group of this moiety is bound to the adjacent O atom in the target compound. However, both reactions lead to the formation of OH groups which are apt to react with respective crosslinking agents in the thermally curable compositions of the invention.

In case of m=1 the one or more species Z—[O(C═O)R¹)ₘ—SH]ₙ can be obtained by reacting one or more species Z[OH]ₙ with n species of HOOC—R¹—SH; wherein Z, R¹, m and n are defined as for the hydroxyl-functional thioether compounds of the invention. However, numerous species of formula Z—[O(C═O)R¹)ₘ—SH]ₙ with m=1 are commercially available from Bruno Bock Chemische Fabrik GmbH & Co. KG, Marschacht, Germany under the tradename Thiocure®.

Typical and preferred species Z[OH]ₙ are e.g., alkylene diols having 2 to 10 carbon atoms such as ethylene glycol, propylene glycol, butane-1,4-diol, pentane-1,5-diol, hexane-1,6-diol, neopentyl glycol, 2-butyl-2-ethylpropane-1,3-diol and dimethylolcyclohexane; diethylene glycol, triethyleneglycol, dipropyleneglycol, tripropyleneglycol, glycerol, diglycerol, trimethylolpropane, trimethylolethane, sugar alcohols, trishydroxyethyl isocyanurate, di(trimethylolpropane), pentaerythritol, and di(pentaerythritol).

Typical and preferred species HOOC—R¹—SH are mercaptoacetic acid, 2-mercaptopropionic acid, 3-mercaptopropionic acid or mercaptobutyric acid such as 3-mercaptobutyric acid.

The one or more species of formula (II) are glycidyl esters of branched or linear alkanoic acids (R³COOH), preferably branched alkanoic acids, R³ being defined as for the hydroxyl-functional thioether compounds of the invention. Preferably, the one or more species of formula (II) are glycidyl esters of trialkyl acetic acid and even more preferred glycidyl esters of neodecanoic acid. Such species are e.g., commercially available under the tradename Cardura® E10 from Hexion, Inc.

The reaction between the one or more species Z—[O(C═O)R¹)ₘ—SH]ₙ and the one or more species of formula (II) is preferably carried out in the presence of an organic solvent which is inert in the reaction. Such solvents are e.g., hydrocarbons such as Solvent Naphtha. Preferably the one or more species Z—[O(C═O)R)ₘ—SH]ₙ are weighed in the reaction container and the one or more species of formula (II) are added slowly while stirring. Preferably the reaction is carried out under inert gas atmosphere such as nitrogen atmosphere. Typically, there is no need of heating the reactions mixture. The reaction is carried out until essentially all thiol groups have been consumed. The reaction can be carried out in the presence of a catalyst, preferably a base as a catalyst such as a tertiary amine or tetramethyl guanidine.

### Thermally Curable Composition

The thermally curable compositions may be one-pack compositions or two-pack compositions. They are preferably solvent-based coating compositions.

The term “one-pack composition”—as defined in the textbook “Rompp Lexikon Lacke und Druckfarben”, Thieme, 1998—refers to compositions, particularly coating compositions, which, contrary to the below described two-pack compositions are produced and supplied in a way that they contain the base resins and the crosslinking agents in one composition without premature reaction between the ingredients. Reaction is preferably caused either by heating or baking.

The term “two-pack composition” as used herein refers to compositions, particularly coating compositions comprising at least two packs, each of which is stable at storage conditions, but which contain ingredients which are, after mixing, reactive with each other. Typically, none of the at least two packs alone is apt to provide a suitable coating, adhesive layer or sealant. In the present invention it is preferred that the compositions are two-pack compositions.

The thermally curable compositions according to the invention comprise one or more hydroxyl-functional thioether compounds of the invention; and one or more crosslinking agents, which are reactive with the hydroxyl groups of the one or more hydroxyl-functional thioether compounds. The thermally curable compositions may further contain additional binders e.g., from the group of polymeric polyol or typical additives. Furthermore, organic solvents and/or colorants and fillers may be contained.

Thermally curable composition is preferably a coating composition, an adhesive composition or a sealant composition, most preferably a coating composition.

### Crosslinking Agents (B)

### Di- and Polyisocyanates

Where the thermally curable compositions of the present invention are two-pack compositions, the isocyanate groups contained in the diisocyanates and/or polyisocyanates are free isocyanate groups.

Most preferred crosslinking agents to be used in the thermally curable two-pack compositions of the present invention are diisocyanates and/or polyisocyanates. Polyisocyanates, as defined herein, have on average more than two free isocyanate groups.

While the diisocyanates and/or polyisocyanates of the present invention can be aromatic polyisocyanates such as tolylene diisocyanates and their dimers, trimers and polymers, it is more preferred to employ aliphatic diisocyanates and/or aliphatic polyisocyanates in the thermally curable two-pack compositions of the invention. The use of aromatic polyisocyanates is less preferred, since coatings obtained from coating materials comprising aromatic polyisocyanates tend to an undesirable yellowing. Therefore, the use of aromatic diisocyanates and/or aromatic polyisocyanates in coating materials is less preferred.

Aliphatic diisocyanates and aliphatic polyisocyanates, respectively, are herein understood to be compounds having at least two free, specifically at least three free, isocyanate groups in the molecule, that is, isocyanate groups which are not blocked at room temperature (25° C.). The term “aliphatic polyisocyanates” also comprehends dimers, trimers and polymers of the aliphatic diisocyanates. Examples thereof are dimers, trimers and polymers of hexamethylene diisocyanate (HDI), including for example its uretdiones and more particularly its isocyanurates and iminooxadiazinediones, the iminooxadiazinediones being most suitable, if a particularly low viscosity is needed.

Herein, the term “aliphatic” also encompasses the term “cycloaliphatic”, such as more particularly isophorone diisocyanate (IPDI) and its dimers, trimers and polymers, or cyclohexane (bis-alkyl isocyanate) and also the dimers, trimers and polymers thereof.

Preferred aliphatic unblocked polyisocyanates are trimers of HDI, for example, as Basonat HI 100 from BASF SE (Ludwigshafen, Germany), as Desmodur® N 3300 and Desmodur® XP 2410 from Bayer Material Science AG (Leverkusen, Germany), or as Tolonate® HDT and HDB from Perstorp AB in Perstorp, Sweden, and also similar products from Asahi Kasei Chemicals, Kawasaki, Japan, trade name Duranate® TLA, Duranate® TKA or Duranate® MHG.

Where the thermally curable compositions of the present invention are one-pack compositions, the isocyanate groups contained in the diisocyanates and/or polyisocyanates are not free, but blocked isocyanate groups. Generally, all of the before-mentioned diisocyanates and polyisocyanates can be used, after reacting the free isocyanate groups with a blocking agent, as so-called blocked diisocyanates and blocked polyisocyanates.

Blocking agents for preparing blocked diisocyanates and polyisocyanates are for example
i. phenols, pyridinols, thiophenols and mercaptopyridines, preferably selected from the group consisting of phenol, cresol, xylenol, nitrophenol, chlorophenol, ethylphenol, t-butylphenol, hydroxybenzoic acid, esters of this acid, 2,5-di-tert-butyl-4-hydroxytoluene, thiophenol, methylthiophenol and ethylthiophenol;
ii. alcohols and mercaptanes, the alcohols preferably being selected from the group consisting of methanol, ethanol, n-propanol, isopropanol, n-butanol, isobutanol, t-butanol, n-amyl alcohol, t-amyl alcohol, lauryl alcohol, ethylene glycol monomethyl ether, ethylene glycol monoethyl ether, ethylene glycol monopropyl ether, ethylene glycol monobutyl ether, diethylene glycol monomethyl ether, diethylene glycol monoethyl ether, diethylene glycol monopropyl ether, diethylene glycol monobutyl ether, propylene glycol monomethyl ether, methoxy methanol, 2-(hydroxyethoxy)phenol, 2-(hydroxypropoxy)phenol, glycolic acid, glycolic esters, lactic acid, lactic esters, methylol urea, methylol melamine, diacetone alcohol, ethylene chlorohydrin, ethylene bromohydrin, 1,3-dichloro-2-propanol, 1,4-cyclohexyldimethanol or acetocyano hydrin, and the mercaptanes preferably being selected from the group consisting of butyl mercaptane, hexyl mercaptane, t-butyl mercaptan, t-dodecyl mercaptane;
iii. oximes, preferably the ketoximes of the groups consisting of the ketoxime of tetramethylcyclobutanedione, methyl n-amyl ketoxime, methyl isoamyl ketoxime, methyl 3-ethylheptyl ketoxime, methyl 2,4-dimethylpentyl ketoxime, methyl ethyl ketoxime, cyclohexanone oxime, methyl isopropyl ketoxime, methyl isobutyl ketoxime, diisobutyl ketoxime, methyl t-butyl ketoxime, diisopropyl ketoxime and the ketoxime of 2,2,6,6-tetramethylcyclohexanone; or the aldoximes, preferably from the group consisting of formaldoxime, acetaldoxime;
iv. amides, cyclic amides and imides, preferably selected from the group consisting of lactams, such as ε-caprolactam, δ-valerolactam, γ-butyrolactam or β-propiolactam; acid amides such as acetoanilide, acetoanisidinamide, acrylamide, methacrylamide, acetamide, stearamide or benzamide; and imides such as succinimide, phthalimide or maleimide;
v. imidazoles and amidines;
vi. pyrazoles and 1,2,4-triazoles, such as 3,5-dimethylpyrazole and 1,2,4-triazole;
vii. amines and imines such as diphenylamine, phenylnaphthylamine, xylidine, N-phenylxylidine, carbazole, aniline, naphthylamine, butylamine, dibutylamine, butylphenylamine and ethyleneimine;
viii. imidazoles such as imidazole or 2-ethylimidazole;
ix. ureas such as urea, thiourea, ethyleneurea, ethylenethiourea or 1,3-diphenylurea;
x. active methylene compounds such as dialkyl malonates like diethyl malonate, and acetoacetic esters; and
xi. others such as hydroxamic esters as for example benzyl methacrylohydroxamate (BMH) or allyl methacrylohydroxamate, and carbamates such as phenyl N-phenylcarbamate or 2-oxazolidone.

### Further Crosslinking Agents

It is also possible to use crosslinkers different from the abovementioned free and blocked diisocyanates and polyisocyanates, respectively. Especially advantageous in this context are those which enter into curing reactions with the reactive diluent or other binders within the same temperature range as the free or blocked diisocyanates and/or free or blocked polyisocyanates, i.e., the range from 20° C. to 200° C. Examples of such crosslinkers include components containing silyl groups, of the kind specified in WO 2008/074489, WO 2008/074490 and WO 2008/074491.

Further preferred crosslinkers for one-pack coating materials are aminoplast crosslinkers having active methylol, methylalkoxy or butylalkoxy groups and blocked polyisocyanate crosslinkers, which are reactive with e.g., hydroxyl groups.

Aminoplasts, or amino resins, are described in Encyclopedia of Polymer Science and Technology vol. 1, p. 752-789 (1985). An aminoplast is obtained by reaction of an activated nitrogen with a lower molecular weight aldehyde, optionally with further reaction with an alcohol (preferably a mono-alcohol with one to four carbon atoms such as methanol, isopropanol, n-butanol, isobutanol, etc.) to form an ether group. Preferred examples of activated nitrogens are activated amines such as melamine, benzoguanamine, cyclohexylcarboguanamine, and acetoguanamine; ureas, including urea itself, thiourea, ethylene urea, dihydroxyethylene urea, and guanyl urea; glycoluril; amides, such as dicyandiamide; and carbamate-functional compounds having at least one primary carbamate group or at least two secondary carbamate groups. The activated nitrogen is reacted with a lower molecular weight aldehyde. The aldehyde may be selected from formaldehyde, acetaldehyde, crotonaldehyde, benzaldehyde, or other aldehydes used in making aminoplast resins, although formaldehyde and acetaldehyde, especially formaldehyde, are preferred. The activated nitrogen groups are at least partially alkylolated with the aldehyde, and may be fully alkylolated; preferably the activated nitrogen groups are fully alkylolated. The reaction may be catalyzed by an acid, e.g. as taught in U.S. Pat. No. 3,082,180, which is incorporated herein by reference.

### Further Polymeric Binders (C)

Besides the one or more hydroxyl-functional thioether compounds of the invention and the crosslinking agents, further polymeric binders, which differ from the afore-mentioned hydroxyl-functional thioether compounds and crosslinking agents, can be contained in the thermally curable compositions of the present invention.

Amongst the further polymeric binders which are preferably contained in the thermally curable compositions of the present invention, hydroxyl-functional polymeric binders, i.e., “polymeric polyols” are particularly preferred. Preferably, the polymeric polyols do not contain sulfur.

A “polymeric polyol” herein means a polyol having at least two hydroxyl groups, the term “polymeric” herein also encompassing the term “oligomeric”. Oligomers herein are composed of at least three monomer units.

Preferably, the polymeric polyols have weight-average molecular weights M, >500 Da, determined by means of GPC (gel permeation chromatography) using a polystyrene standard, specifically of 800 to 100,000 Da, more particularly of 1,000 to 50,000 Da. Even more preferred, the polymeric polyols are those having a weight-average molecular weight of 1,000 to 10,000 Da.

Preferably, the polymeric polyols have a hydroxyl number (OH number) of 30 to 350 mg KOH/g, more preferred of 80 to 250 mg KOH/g, and particularly preferred of 100 to 180 mg KOH/g. The hydroxyl number indicates the number of mg of potassium hydroxide that are equivalent to the amount of acetic acid bound by 1 g of substance on acetylation. The sample is boiled with acetic anhydride-pyridine for the determination, and the resulting acid is titrated with potassium hydroxide solution (DIN 53240-2).

The polymeric polyols are preferably selected from the group comprising or consisting of poly(meth)acrylic polyols, polyester polyols, polyether polyols, polyether-polyester polyol and polyurethane polyols.

Preferably the polymeric polyols are poly(meth)acrylate polyols. As used herein, the term “poly(meth)acrylate” refers to not only polyacrylates but also polymethacrylates, and also polymers which comprise both methacrylates and/or methacrylic acid and acrylates and/or acrylic acid. Besides acrylic acid, methacrylic acid and/or the esters of acrylic acid and/or methacrylic acid, the poly(meth)acrylates may also comprise other ethylenically unsaturated monomers. In one or more embodiments, the monomers from which the poly(meth)acrylates are obtained are monoethylenically unsaturated monomers. A “poly(meth)acrylate polyol” refers to a poly(meth)acrylate which contains at least two hydroxyl groups. They may be prepared in a single stage or a multiplicity of stages. They may also be present in the form, for example, of random polymers, gradient copolymers, block copolymers or graft polymers.

The hydroxyl groups in the poly(meth)acrylate polyols are introduced in the polymer by use of hydroxyl-functional monomers in the polymerization reaction. Hydroxyl-containing monomers of the poly(meth)acrylate polyols that are used are preferably selected from hydroxyalkyl(meth)acrylates, such as, more particularly, 2-hydroxyethyl acrylate, 2-hydroxyethyl methacrylate, 2-hydroxypropyl acrylate, 2-hydroxypropyl methacrylate, 3-hydroxypropyl acrylate, 3-hydroxypropyl methacrylate, 3-hydroxybutyl acrylate, 3-hydroxybutyl methacrylate, and also, in particular, 4-hydroxybutyl acrylate and/or 4-hydroxybutyl methacrylate. In particular, it is also possible with advantage to use mixtures resulting from the industrial preparation. Thus, for example, industrially prepared hydroxypropyl methacrylate is composed of about 20%-30% 3-hydroxypropyl methacrylate and 70%-80% 2-hydroxypropyl methacrylate.

As further monomers for the synthesis of the poly(meth)acrylate polyols it is possible to use alkyl(meth)acrylates, such as, ethyl acrylate, ethyl methacrylate, propyl acrylate, propyl methacrylate, isopropyl acrylate, isopropyl methacrylate, butyl acrylate, butyl methacrylate, isobutyl acrylate, isobutyl methacrylate, tert-butyl acrylate, tert-butyl methacrylate, amyl acrylate, amyl methacrylate, hexyl acrylate, hexyl methacrylate, ethylhexyl acrylate, ethylhexyl methacrylate, 3,3,5-trimethylhexyl acrylate, 3,3,5-trimethylhexyl methacrylate, stearyl acrylate, stearyl methacrylate, lauryl acrylate or lauryl methacrylate, cycloalkyl acrylates and/or cycloalkyl methacrylates, such as cyclopentyl acrylate, cyclopentyl methacrylate, isobornyl acrylate, isobornyl methacrylate or, in particular, cyclohexyl acrylate and/or cyclohexyl methacrylate.

As further monomers for the synthesis of the poly(meth)acrylate polyols it is possible to use vinylaromatic hydrocarbons, such as vinyltoluene, alpha-methylstyrene or, in particular, styrene, amides or nitriles of acrylic or methacrylic acid, vinyl esters or vinyl ethers, and also, in minor amounts, in particular, acrylic and/or methacrylic acid.

It is further possible to use poly(meth)acrylates polyols, prepared by use of hydroxy or carboxyl group containing monomers where at least part of the OH and/or COOH groups of the polymerized monomer were modified with mono-oxirane group containing monomer such as the species of formula (II) as defined above. Such modifications are e.g., described in Z. W. J. Wicks, F. N. Jones, S. P. Pappas and D. A. Wicks, Organic Coatings—Science and technology, Hoboken, New Jersey: Wiley, 2007; or H. Petit, N. Henry, A. Krebs, G. Uytterhoeven and F. de Jong, “Ambient cure high solids acrylic resins for automotive refinish clear coat applications,” Progress in Organic Coatings, pp. 41-49, 2001; and WO 2003/011923 A1 (see examples at page 8 and following).

Instead of or in addition to the poly(meth)acrylate polyols, it is also possible to use polyester polyols. A polyester polyol here is a polyester which carries at least two hydroxyl groups.

In the case of joint use of poly(meth)acrylate polyols and polyester polyols, both components may be prepared individually or by polymerizing the poly(meth)acrylate polyol in situ in a polyester polyol component or solution thereof in an appropriate solvent.

Hydroxyl groups in such poly(meth)acrylic polyols can be further modified by at least partially reacting these with glycidiyl esters, such as e.g., compound of the above formula (II). Also, urea-crystal-modified poly(meth)acrylic polyols can be employed.

Suitable polyester polyols are described in EP-A-0 994 117 and EP-A-1 273 640, for example. In particular, it is possible to obtain suitable polyester polyols, as is known to a person of ordinary skill in the art, through polycondensation from polyols and polycarboxylic acids or their anhydrides.

Particularly suitable as polyol or polyol mixture which can be used in the polycondensation reaction to produce polyester polyols are polyhydric alcohols and mixtures thereof, the alcohols having at least two, preferably at least three, hydroxyl groups. In one or more embodiments, the polyol mixture used or the polyol used comprises or is at least one polyfunctional polyol containing at least three hydroxyl groups. Suitable polyfunctional polyols having at least three hydroxyl groups are selected from the group consisting of trimethylolpropane (TMP), trimethylolethane (TME), glycerol, pentaerythritol, sugar alcohols, ditrimethylolpropane, dipentaerythritol, diglycerol, trishydroxyethyl isocyanurate and mixtures thereof. In one specific embodiment the polyol used for preparing the polyester polyols is composed only of polyfunctional polyols having more than three hydroxyl groups. In another specific embodiment, the polyol mixture used for preparing the polyester polyols comprises at least one polyfunctional polyol having at least three hydroxyl groups and at least one diol. Examples of suitable diols include ethylene glycol, propylene glycol, butane-1,4-diol, pentane-1,5-diol, hexane-1,6-diol, neopentyl glycol, 2-butyl-2-ethylpropane-1.3-diol, diethylene glycol, dipropylene glycol, higher polyether diols, dimethylolcyclohexane, and mixtures of the aforementioned polyols.

Polycarboxylic acids or anhydrides thereof that are suitable for preparing the polyester polyols are, for example, phthalic acid, phthalic anhydride, isophthalic acid, terephthalic acid, trimellitic acid, trimellitic anhydride, pyromellitic acid, pyromellitic dianhydride, tetrahydrophthalic acid, 1,2-, 1,3- or 1,4-cyclohexanedicarboxylic acid, 1,2-cyclohexanedicarboxylic anhydride, tricyclodecanedicarboxylic acid, succinic acid, glutaric acid, adipic acid, pimelic acid, suberic acid, azelaic acid, sebacic acid, maleic acid, maleic anhydride, fumaric acid, itaconic acid, mesaconic acid, citraconic acid, dimer fatty acids and mixtures thereof.

Where the coating material composition of the invention further comprises additional binders, apart from the binders which may be subsumed under the term of the polymeric polyols, then these other binders may react with the other components of the coating material or else be chemically inert with respect to said components.

Such binders may include physically drying binders, that is, binders that are inert chemically with respect to the other paint constituents, as e.g., cellulose acetobutyrate (CAB), polyamides or polyvinyl butyral.

### Additives

Furthermore, the thermally curable compositions may also comprise further typical additives, particularly additives for coating material which different from the afore-mentioned components. Any of the additives differs from the herein afore-mentioned binders (A), (B) and (C).

Examples of suitable coatings additives are:
in particular, UV absorbers such as, for example, 2-(2-hydroxyphenyl) benzotriazoles, 2-hydroxybenzophenones, hydroxyphenyl-s-triazines, and oxalanilides;
in particular, light stabilizers such as those known as HALS compounds (“hindered amine light stabilizers”; these are derivatives of 2,2,6,6-tetramethylpiperidine; available commercially for example as Tinuvin® 292 from BASF SE), benzotriazoles such as hydroxyphenylalkylbenzotriazole, or oxalanilides;
radical scavengers;
slip additives;
polymerization inhibitors;
defoamers;
reactive diluents, more particularly reactive diluents which become reactive only through reaction with other constituents and/or with water, such as oxazolidines (such as Incozol®) or aspartic esters, for example;
wetting and dispersing agents, such as silxoanes, fluorine-containing compounds, carboxylic monoesters, phosphoric esters, polyacrylic acids and copolymers thereof, or polyurethanes;
adhesion promoters;
flow control agents, especially those based on a polyacrylate. Employed preferably here are copolymers of ethylhexyl acrylate and ethyl acrylate. These copolymers preferably have a very low TG, are relatively nonpolar, and have a low OH number;
film-forming assistants such as cellulose derivatives;
rheology control additives, such as the additives known from patents WO 94/22968, EP-A-0 276 501, EP-A-0 249 201, or WO 97/12945; crosslinked polymeric microparticles, as disclosed for example in EP-A-0 008 127; inorganic phyllosilicates such as aluminum magnesium silicates, sodium magnesium phyllosilicates and sodium magnesium fluorine lithium phyllosilicates of the montmorillonite type; silicas such as Aerosils®; or synthetic polymers having ionic and/or associative groups such as poly(meth)acrylamide, poly(meth)acrylic acid, polyvinylpyrrolidone, styrene-maleic anhydride copolymers or ethylene-maleic anhydride copolymers and their derivatives, or hydrophobically modified ethoxylated urethanes, or polyacrylates; and
flame retardants.

### Colorants and Fillers

The thermally curable compositions of the invention may further comprise colorants such as soluble dyes, pigments or fillers. If the thermally curable compositions are used as clearcoat coating compositions, they are free or substantially free from nontransparent pigments and fillers. However, fillers in the form of nanoparticles based on silicon dioxide, aluminum oxide, or zirconium oxide may be used (for further details it is referred to Römpp Lexikon “Lacke und Druckfarben” Georg Thieme Verlag, Stuttgart, 1998, pages 250 to 252).

### Solvents

In particular the compositions may also comprise solvents. Suitable solvents include all typical paint solvents such as, more particularly, aromatic hydrocarbons or butyl acetate.

The solvents are preferably polar organic solvent and/or aromatic solvents. Among useful solvents are ketones, esters, acetates, aprotic amides, aprotic sulfoxides, and aprotic amines, aliphatic and/or aromatic solvents. Examples of specific useful solvents include ketones, such as acetone, methyl ethyl ketone, methyl amyl ketone, methyl isobutyl ketone, esters such as ethyl acetate, butyl acetate, pentyl acetate, ethyl ethoxypropionate, ethylene glycol butyl ether acetate, propylene glycol monomethyl ether acetate, aliphatic and/or aromatic hydrocarbons such as toluene, xylene, solvent naphtha, and mineral spirits, ethers such as glycol ethers like propylene glycol monomethyl ether, alcohols such as ethanol, propanol, isopropanol, n-butanol, isobutanol, and tert-butanol or alkoxyalkanoles, such as methoxypropanol or di(propyleneglycol) monomethyl ether, nitrogen-containing compounds such as N-methyl pyrrolidone and N-ethyl pyrrolidone, and combinations of these.

### Amounts and Ratios of Components in the Thermally Curable Composition

Preferably, hydroxyl-functional thioether compounds of the invention are present in the thermally curable compositions of the invention in an amount of 0.1% to 70% by weight, more preferred in an amount of 1% to 50% by weight, most preferred in an amount of 2% to 30% by weight or even better 3% to 15% by weight, based on the total weight of binders in the composition. The amount of binders (i.e., the binder content) is the solids content of the composition minus the amount of pigments and fillers. The solids content is determined as the fraction of 1 g of the composition left after drying the composition for 60 min at 130° C. The amount of pigments and fillers is the weighed dry amount of pigments and fillers employed in the composition. In pigment free and filler free compositions, such as clear coat compositions the binder content equals the solids content.

The amount ranges of the other ingredients, such as the crosslinking agents (B), the further polymeric binders (C), the additives, colorants, fillers and solvents are not particularly critical. Typically, the amount range of crosslinkers (B) results from the chosen amount of the hydroxyl-functional thioether compounds of the invention and the further polymeric binders (C). If the coating compositions e.g., comprise pigments and fillers, additives such as wetting and dispersing agents may need to be used to keep the pigments and fillers dispersed. Since the hydroxyl-functional thioether compounds of the invention also act as solvents (reactive diluents), typically the amounts of non-reactive solvents can be varied and preferably reduced.

Where the fraction of the hydroxyl-functional thioether compounds of the invention is below 0.1% by weight, based on the total weight of binders, the effect according to the invention is usually small.

The thermally curable two-pack compositions of the invention are preferably prepared shortly before their application, by mixing of the components, since a crosslinking reaction between the reactive groups of the crosslinking agent, preferably the free isocyanate groups of the diisocyanates and/or polyisocyanates and the hydroxyl groups present in the hydroxyl-functional thioether compounds of the invention and the optional polymeric polyols may proceed even at room temperature. Preliminary mixing of the hydroxyl-functional thioether compounds and the polymeric polyols, if present, generally presents no problems. In each case, the constituents of the curable composition that are reactive with one another ought to be mixed not until shortly before the application of the composition, such as the coating composition, adhesive or sealant in order to ensure that as less undesired reactions as possible take place.

### Use of the Hydroxyl-Functional Thioether Compound

The hydroxyl-functional thioether compound of the invention can be used as a reactive diluent in thermally curable compositions, the compositions preferably being selected from the group of coating compositions, adhesives and sealants. Therefore, this use is a further object of the present invention.

If used in a coating composition, the coating composition of the invention is preferably employed as the uppermost paint coat in a multilayer coating. Particularly preferred, it is applied as a clearcoat in automotive coating. Since the compositions of the invention cure chemically even at low temperatures they can be used in automotive refinishing.

### Multilayer Coating, Method of Producing the Same and Thus Coated Substrate

The present invention further provides a multilayer coating comprising at least two coating layers, preferably at least three coating layers. The coating layers are disposed on a primed or unprimed substrate, the base coat and/or preferably the uppermost coat being formed from a thermally curable coating composition of the invention. If the substrate is primed, then the primer, is preferably an electrodeposition primer, more particularly a cathodic electrodeposition primer. Priming may also, in particular, be preceded by a conversion coating step such as a phosphatizing treatment to form a conversion coating layer.

Atop the primed or unprimed substrate there may be, for example, an applied conventional primer-surfacer coating material. Onto the primer-surfacer coating material, if present, there may be one or more basecoats applied, which might be formed from any conventional basecoat compositions. In the case of automotive refinishing, the basecoats in question are typically those which dry purely physically or those which cure thermally, by means of a crosslinking agent, or those which cure thermally and actinically, or actinically only. Another possibility is to furnish primer-surfacer coating materials with the properties of a basecoat or, conversely, to furnish a basecoat with primer-surfacer properties, with the consequence that it may be sufficient to apply only one primer-surfacer coat or only one basecoat. Typically, a primer-surfacer coating material is applied as primer-surfacer coat, and at least one basecoat composition is applied as basecoat. Suitability as primer-surfacer coating material and as basecoat composition is possessed by all commercial primer-surfacers or basecoat materials, more preferred those as used in automotive refinishing. While it is possible that the basecoat material is a thermally curable composition according to the present invention, it is more preferred, that the last coat applied is a thermally curable coating composition of the invention as a topcoat, specifically as a transparent topcoat (clearcoat).

Additionally, provided by the invention, is a method for producing a multilayer coating of the invention, comprising the following steps:
(i) applying at least one basecoat composition on an untreated or pretreated, optionally electrodeposition coated and/or primer-surfacer coated substrate to form a basecoat layer and subsequently
(ii) applying at least one topcoat composition, preferably in the form of a clearcoat compositions onto the basecoat composition to form a topcoat layer, preferably a clearcoat layer, followed by
(iii) curing of not fully cured coating layers at a temperature in the range from 20° C. to 200° C.,
   wherein at least one basecoat composition or at least one topcoat composition is a thermally curable composition according to the invention. Preferably, only the topcoat composition is a thermally curable composition according to the invention.

As primer-surfacer coating material and basecoat composition it is possible to use conventional, commercial primer-surfacers and basecoat compositions.

The individual coating layers are applied by the customary painting methods familiar to a person of ordinary skill in the art. The compositions and coating materials are preferably applied by spraying, pneumatically and/or electrostatically.

The application of the basecoat composition on a primer-surfacer layer and/or the application of the topcoat composition on the basecoat layer may be carried out by a wet-on-wet application, i.e., an application where the preceding layer is not cured or not fully cured before the coating composition is applied. Thus, prior to the application of the basecoat composition or basecoat compositions, the primer-surfacer may merely be flashed off at room temperature, or else, alternatively, it may be dried at elevated temperature. Drying may also take place by IR irradiation.

The comments made above in relation to the primer-surfacer coating system, with regard to the flashing and/or drying, also apply equally to the basecoat or basecoats.

Instead of a wet-on-wet application, a further possibility is the curing of the primer-surfacer layer and/or basecoat layer or any other coating layers prior to the application of the topcoat composition. The conventional primer-surfacers and/or conventional basecoat composition can be cured thermally, with actinic radiation, or with a combination of thermal curing and actinic radiation curing.

The curing of the coating layer formed from a thermally curable composition of the invention, e.g., a basecoat layer or clearcoat layer takes place at temperatures up to 200° C., more specifically at temperatures from room temperature (i.e., 25° C.) to 150° C., and very specifically at temperatures from 30 to 100° C.

In case the thermally curable composition of the invention is a one-pack composition, the curing temperature is preferably from above 100° C. to 200° C., more preferred from 110 to 190° C., and most preferred from 120 to 180° C., for a time of 5 min up to 30 min, and more preferably 10 min up to 25 min.

In case the thermally curable composition of the invention is a two-pack composition, the curing temperature is preferably from 20° C. to 130° C. more preferred from 40 to 100° C., and most preferred from 60 to 90° C., for a time of 5 min up to 30 min, and more preferably 10 min up to 25 min.

Additionally, provided by the present invention is a substrate coated with a multilayer coating of the invention.

Suitable substrate materials include, in particular, metallic substrates, such as automotive bodies and parts thereof, but also plastics substrates can be used, preferably those being used in automotive production.

### Examples

In the following the present invention is further explained by means of working examples and comparative examples. Amounts given in the tables below are amounts by weight or in weight percent, unless specified otherwise.

### Testing Procedures

### Solids Content

The solids content was measured according to DIN EN ISO 3251 by drying approx. 1 g of the sample for 60 minutes at 130° C.

### Sagging

Determination of sagging resistance was done through an application of the coating material in a wedge with a layer thickness from 10 to 50 μm onto panels primed with an electrodeposited coating and coated with a basecoat. The basecoat was cured before application of the clearcoat formulation. Sagging occurred on holes in the panels, imitating edges in a car body. Measurand for sagging was the layer thickness, at which sagging length reached 3 mm and 10 mm length.

In more detail, a perforated steel panel having dimensions of 57 cm×20 cm (according to DIN EN ISO 28199-1, section 8.1, version A), coated with a cured cathodic electrodeposition paint (Cathoguard® 800 from BASF Coatings GmbH) and with a cured commercially available water-based basecoat material (ColorBrite from BASF Coatings GmbH) was prepared analogously to DIN EN ISO 28199-1, section 8.2 (version A). The clearcoat material was then electrostatically applied in the form of wedges with a target film thickness (film thickness of the dried material) of 10 μm to 50 μm in a single application in a method based on DIN EN ISO28199-1, section 8.3. After a flash time of 10 minutes at room temperature (25° C.), the resulting clear coating film was cured in a forced air oven at 80° C. for 30 minutes. The steel panels were flashed and cured while standing upright.

The sagging was determined in each case according to DIN EN ISO 28199-3, section 4. Measurand for sagging was the layer thickness, at which sagging length reached 3 mm and 10 mm length.

### Levelling

Measurands for levelling were values obtained through measurements with the Byk WaveScan. Longwave values should imitate the visual appearance of structures with a wavelength of 1.2 to 12 mm. Shortwave value should imitate the visual appearance of structures with a wavelength of 0.3 to 1.2 mm. The measurements were carried out at 37.5±2.5 μm film thickness of the cured coating.

### Chemical Resistance

Chemical resistance was tested according to DIN EN ISO 2812-5 (December 2018), for tree gum, a 1% sodium hydroxide solution and a 1% sulfuric acid solution. Testing temperature was 36 to 78° C., testing time was 15 minutes.

### Scratch Resistance

Scratch resistance was tested with an AMTEC-Kistler device, imitating an industrial carwash facility. 20° gloss was measured before the test, after a cleaning of the panels after the test and again after a 2 hour reflow at 60° C. DIN EN ISO 20566 DE (2013). Gloss was measured according to DIN EN ISO 2813 (2015).

### Materials

| Abbreviation | Chemical Name |
|---|---|
| PETMP | pentaerythritol tetrakis(3-mercaptopropionate) |
| TMPMP | trimethylol propane tris(3-mercaptopropionate) |
| DiPETMP | di-pentaerythritol hexakis(3-mercaptopropionate) |
| GDMA | ethylene glycol bis(3-mercaptoacetate) |
| GDMP | ethylene glycol bis(3-mercaptopropionate) |
| CRD | comparative reactive polyacrylate diluent (OH value |
| | of solids: 130 mg KOH/g; solids content: 66.5 wt.-%) |
| Cardura ® | glycidyl neodecanoate |
| E-10 | |
| Desmodur ® | hexamethylene diisocyanate isocyanurate (NCO content |
| N 3300 | 21.8 ± 0.3 wt.-%; equivalent weight ≈ 193) |
| Acrylic | polyacrylate (OH value of solids: 150 mg KOH/g; solids |
| Binder 1 | content: 60 wt.-%) |
| Acrylic | urea-crystal modified polyacrylate (OH value of solids: |
| Binder 2 | 106 mg KOH/g; solids content: 59 wt.-%) |
| Surface | polyethermodified polydimethylsiloxane |
| Additive | (solids content: 50 wt.-%) |
| UV Absorber | hydroxyphenyl-triazine type UV stabilizer |
| | (solids content: 95 wt.-%) |
| Light | hindered amine type light stabilizer (solids content: 100 |
| Stabilizer | wt.-%) |
| Catalyst | bismuth neodecanoate (solids content: 100 wt.-%) |
| Conductivity | alkylol ammonium salt of unsaturated carboxylic acid |
| Agent | ester (solids content: 80 wt.-%) |
| Solvent | proprietory mixture of solvents comprising butyl acetate, |
| Package | butyl glycol acetate, white spirits and ethyl ethoxy |
| | propionate |
| SN II | Solvent Naphtha II |

### Manufacture of Hydroxyl-Functional Thioether Compounds A1 to A5

The hydroxyl-functional thioether compounds were synthesized by reacting the thiols as indicated in Table 1 with glycidyl neodecanoate (Cardura® E-10) in the presence of tetramethylguanidine as a catalyst, based on the solids content of the mixture. All amounts are parts by weight. The reaction was carried out by placing the thiol and part of SN II (were used), under nitrogen atmosphere, in a 3-neck glass flask equipped with a temperature control device and a reflux condenser. Over the course of 45 min the glycidyl neodecanoate was added dropwise through a dropping funnel under stirring, without heating. Any glycidyl neodecanoate remaining in the dropping funnel after 45 min was flushed into the reaction mixture using the residual amount of SN II. The maximum reaction temperature (Tₘₐₓ) reached in the course of the reaction is also indicated in Table 1.

TABLE 1

| | | | | Hydroxyl-functional thioether compound | | | | |
|---|---|---|---|---|---|---|---|---|
| Reactants | n | R¹ | R³ | A1 | A2 | A3 | A4 | A5 |
| PETMP | 4 | CH₂CH₂ | ./. | 27.40 | — | — | — | — |
| TMPMP | 3 | CH₂CH₂ | ./. | — | 27.96 | — | — | — |
| DiPETMP | 6 | CH₂CH₂ | ./. | — | — | 27.59 | — | — |
| GDMA | 2 | CH₂ | ./. | — | — | — | 24.48 | — |
| GDMP | 2 | CH₂CH₂ | ./. | — | — | — | — | 33.25 |
| glycidyl | ./. | ./. | branched | 52.80 | 51.88 | 52.25 | 55.36 | 66.25 |
| neodecanoate | | | C₉-alkyl | | | | | |

| | | | | | |
|---|---|---|---|---|---|
| catalyst | 0.20 | 0.20 | 0.20 | 0.20 | 0.50 |
| SN II | 19.96 | 19.96 | 19.96 | 19.96 | — |
| Tₘₐₓ [° C.] | 47 | 32 | 41 | 62 | 73 |
| solids content (1 h, 130° C.) | 78.70 | 78.00 | 77.90 | 76.80 | 97.20 |
| calculated OH value of solids content | 152.8 | 148.5 | 149.4 | 160.2 | 154.1 |
| [mg KOH/g] | | | | | |

### Manufacture of the Comparative Reactive Diluent (CRD)

A stainless steel reactive in pressure design, equipped with two feed vessels, a reflux condenser, and a stirring member, is charged with solvent naphtha. One of the feed vessels is charged with the monomer mixture of 1340 g 2-Ethylhexylacrylate and 495 g 2-Hydroxyethylacrylate. The second flask is charged with a solution of a radical initiator (e.g. di-tert-butyl peroxide) in solvent naphtha. At a pressure of 2.5 bar absolute, the reactor charge is heated to 150° C. When the temperature is reached, the initiator feed is started; the overall feed time is 270 minutes. 5 minutes after the start of the initiator feed, the monomer feed is commenced, and is fed in over 240 minutes. After the end of both feeds, the batch is held at 150° C. for a further 60 minutes, after which it is cooled down and let down. The solids content of the resin solution is adjusted with solvent naphtha to 66.5%±1%.

### Manufacture of the Thermally Curable Coating Compositions

The thermally curable compositions were obtained by homogeneously mixing pack A (i.e., the crosslinkable binder pack) with pack B (i.e., the crosslinking agent comprising binder pack).

Pack A was obtained by homogeneously mixing the ingredients as indicated in Table 2A. The numbers given are in parts by weight and the ingredients used as well as their solids content are found in the “Materials” section of the present invention.

TABLE 2A

| | | Coating Compositions Pack A | | | | | |
|---|---|---|---|---|---|---|---|
| | | Examples/Comparative Example | | | | | |
| A-Pack | | E1 | E2 | E3 | E4 | E5 | C1 |
| Acrylic Binder 1 | | 45.00 | 45.00 | 45.00 | 45.00 | 45.00 | 45.00 |
| Acrylic Binder 2 | | 11.60 | 11.60 | 11.60 | 11.60 | 11.60 | 11.60 |
| Reactive | A1 | 8.45 | — | — | — | — | — |
| Diluent | A2 | — | 8.55 | — | — | — | — |
| | A3 | — | — | 8.55 | — | — | — |
| | A4 | — | — | — | 8.65 | — | — |
| | A5 | — | — | — | — | 6.85 | — |
| | CRD | — | — | — | — | — | 10.00 |

| | | | | | | |
|---|---|---|---|---|---|---|
| Surface Additive | 0.16 | 0.16 | 0.16 | 0.16 | 0.16 | 0.16 |
| UV Absorber | 1.23 | 1.23 | 1.23 | 1.23 | 1.23 | 1.23 |
| Light Stabilizer | 0.92 | 0.92 | 0.92 | 0.92 | 0.92 | 0.92 |
| Catalyst | 0.02 | 0.02 | 0.02 | 0.02 | 0.02 | 0.02 |
| Conductivity Agent | 0.46 | 0.46 | 0.46 | 0.46 | 0.46 | 0.46 |
| Solvent Package | 28.80 | 28.80 | 28.80 | 28.80 | 28.80 | 28.80 |
| Butyl acetate | 5.70 | 5.80 | 5.80 | 7.00 | 5.60 | 7.60 |
| Calculated OH | 101 | 100 | 99 | 101 | 101 | 97 |
| groups in 100 | | | | | | |
| parts A-Pack | | | | | | |
| [mmol] | | | | | | |
| Reactive diluent | 15.4 | 15.5 | 15.5 | 15.4 | 15.4 | 15.4 |
| solids on total | | | | | | |
| solids content | | | | | | |
| [wt.-%] | | | | | | |
| Viscosity ISO4 flow | 43 | 42 | 43 | 41 | 41 | 43 |
| cup at 23° C. [s] | | | | | | |

Pack B was obtained by homogeneously mixing the ingredients as indicated in Table 2B3. The numbers given are in parts by weight and the ingredients used as well as their solids content are found in the “Materials” section of the present invention.

TABLE 2B

| | Coating Compositions Pack B | | | | | |
|---|---|---|---|---|---|---|
| | Examples/Comparative Example | | | | | |
| B-Pack | E1 | E2 | E3 | E4 | E5 | C1 |
| Desmodur ® N | 70.00 | 70.00 | 70.00 | 70.00 | 70.00 | 70.00 |
| 3300 | | | | | | |
| Butyl acetate | 30.00 | 30.00 | 30.00 | 30.00 | 30.00 | 30.00 |
| Calculated | 106 | 106 | 106 | 106 | 106 | 106 |
| NCO groups | | | | | | |
| in 30 parts B- | | | | | | |
| Pack [mmol] | | | | | | |

In Table 2C the mixing ratio of the A-pack and B-pack as well as the determined solids content of the final coating composition is found.

TABLE 2C

| | Coating Compositions | | | | | |
|---|---|---|---|---|---|---|
| | Examples/Comparative Example | | | | | |
| Two Pack | E1 | E2 | E3 | E4 | E5 | C1 |
| A-Pack | 100.00 | 100.00 | 100.00 | 100.00 | 100.00 | 100.00 |
| B-Pack | 30.00 | 30.00 | 30.00 | 30.00 | 30.00 | 30.00 |
| Solids content | 50.2 | 50.2 | 49.7 | 49.8 | 50.1 | 49.1 |
| (1 h/130° C.) | | | | | | |

### Application of the Thermally Curable Coating Compositions

Except for the levelling and sagging panels, a black basecoat and the clearcoats of Examples E1 to E5 and Comparative Example C1 were applied by pneumatic spray application onto electrodeposition coated steel panels. After 10 min of flash-off, the basecoat was stoved for 10 min at 80° C. The clearcoats were cured for 30 min at 80° C. after a 10-minute flash-off. The clearcoat layer thickness was 30±5 μm.

For levelling and sagging tests, the black basecoat was applied on large electrodeposition coated steel panels in a constant layer thickness. After 10 min flash-off, the basecoat was stoved for 10 min at 80° C. The clearcoats of Examples E1 to E5 and Comparative Example C1 were applied in a wedge-layer thickness from 10 μm to 50 μm thickness by electrostatically application with rotary atomizers. After a vertical flash-off for 10 min the clearcoats were cured vertically for 30 min at 80° C. Additionally to the vertically cured panels, levelling was also measured on panels being flashed-off and cured in a horizontal position (same flash-off and curing conditions).

### Results

All examples—inventive and comparative examples—showed an excellent chemical resistance. With respect to tree gum, the temperature at which the first effects on the coatings were found, was 36° C. for all examples. The respective temperature for all examples with respect to sulfuric acid (1 wt.-% in water) was 55±1° C. and for sodium hydroxide (1 wt.-% in water) 55.5±1.5° C.

All examples—inventive and comparative examples—showed an excellent gloss before carrying out the scratch testing. The gloss for all examples was in the range from 89.5±0.5 gloss units. After a cleaning of the panels after the test, the gloss units for all examples were in the range from 72.5±3.5 and again after a 2 hour reflow at 60° C. the gloss values were 73.5±2.5, thus suggesting a well-balanced reflow behavior.

Consequently, chemical resistance as well as scratch resistance can perfectly compete with the compositions containing the comparative sulfur-free reactive diluent.

However, significant improvements compared to the comparative example C1 were found for inventive examples E1 to E5 with respect to their sagging behavior, while maintaining an excellent levelling behavior. The results are shown in Table 3.

TABLE 3

| | Coating Compositions Pack B | | | | | |
|---|---|---|---|---|---|---|
| | Examples/Comparative Example | | | | | |
| Sagging | E1 | E2 | E3 | E4 | E5 | C1 |
| Thickness at 3 mm | 38 | 37 | 39 | 40 | 40 | 33 |
| runner-length [μm] | | | | | | |
| Thickness at 10 mm | 52 | 51 | 52 | 51 | 52 | 46 |
| runner-length [μm] | | | | | | |
| Levelling | E1 | E2 | E3 | E4 | E5 | C1 |
| Short-wave value (vertical) | 15 | 15 | 13 | 12 | 13 | 15 |
| Short-wave value | 13 | 14 | 14 | 15 | 13 | 14 |
| (horizontal) | | | | | | |
| Long-wave value (vertical) | 13 | 13 | 12 | 11 | 12 | 12 |
| Long-wave value | 7 | 9 | 8 | 9 | 8 | 7 |
| (horizontal) | | | | | | |

The higher the thickness values in the sagging test, the better the sagging resistance. The thickness at 3 mm runner-length is approx. 38.5±1.5 μm compared to 33 μm of the comparative example, which is a significant improvement of approx. 17% in layer thickness. At 10 mm runner-length the thickness value is about approx. 51.5±0.5 μm compared to only 46 μm for the comparative example, still being an improvement of approx. 12%.

The smaller the short-wave and long-wave values, the better the levelling. The average of all short-wave values (vertical) is about 13.5±1.5, while it is 15 for the comparative example; and the average of all short-wave values (horizontal) is about 14±1, thus about the same as for the comparative example, which is 14. The average of all long-wave values (vertical) is about 12±1, and thus about the same as for the comparative example, which is 12; and the average of all long-wave values (horizontal) is about 8±1, while the value for the comparative example is 7.

1. A hydroxyl-functional thioether compound having formula (I) 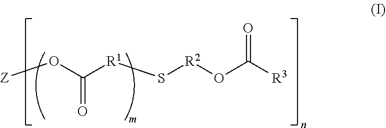
wherein
Z is an n-valent hydrocarbyl group, optionally comprising one or more moieties selected from the group consisting of an ether moiety, a thioether moiety and an isocyanurate moiety;
R¹ is a linear or branched alkylene group;
R² is selected from the group consisting of CH₂CH(OH)CH₂ and CH(CH₂OH)CH₂;
R³ is a linear or branched alkyl group;
m=0 or 1; and
n=2 to 10.

2. The hydroxyl-functional thioether compound according to claim 1, wherein
the n-valent hydrocarbyl group Z contains 2 to 16 carbon atoms; and/or
the linear or branched alkylene group R¹ contains 1 to 6 carbon atoms; and/or
the linear or branched alkyl group R³ contains 1 to 16 carbon atoms; and/or
n=2 to 8.

3. The hydroxyl-functional thioether compound according to claim 1, wherein
the n-valent hydrocarbyl group Z contains 2 to 10 carbon atoms; and/or
the linear or branched alkylene group R¹ contains 1 to 3 carbon atoms; and/or
the linear or branched alkyl group R³ contains 1 to 10 carbon atoms.

4. The hydroxyl-functional thioether compound according to claim 1, wherein
the n-valent hydrocarbyl group Z contains 2 to 10 carbon atoms; and/or
R² is CH₂CH(OH)CH₂; and/or
the linear or branched alkyl group R³ contains 1 to 9 carbon atoms; and/or
n=2 to 6.

5. The hydroxyl-functional thioether compound according to claim 1, wherein
Z is an n-valent hydrocarbyl group or an n-valent hydrocarbyl group comprising one or more ether moieties; and
m=1.

6. The hydroxyl-functional thioether compound according to claim 1, wherein
Z is an n-valent hydrocarbyl group comprising one or more thioether moieties; and m=0.

7. A method of producing a hydroxyl-functional thioether according to claim 1, wherein
(a) one or more species Z—[O(C═O)R)ₘ—SH]ₙ are reacted with
(b) one or more species of formula (II) 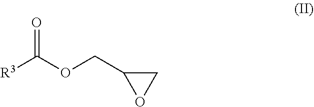
and
(c) that in case of m=1 the one or more species Z—[O(C═O)R)ₘ—SH]ₙ are obtained by reacting one or more species Z[OH]ₙ with n species of HOOC—R¹—SH.

8. A thermally curable composition, comprising
(A) one or more hydroxyl-functional thioethers according to claim 1; and
(B) one or more crosslinking agents, which are reactive with the hydroxyl groups of the one or more hydroxyl-functional thioethers (A).

9. The thermally curable composition according to claim 8, wherein at least one of the crosslinking agents is selected from the group of free diisocyanates, blocked diisocyanates, free polyisocyanates, blocked polyisocyanates, and aminoplast resins.

10. The thermally curable composition according to claim 8, further comprising (C) one or more polymeric polyols.

11. The thermally curable composition according to claim 8, wherein the composition is selected from the group consisting of a coating composition, an adhesive composition, and a sealant composition.

12. A method of using the hydroxyl-functional thioether according to claim 1, wherein the method comprising using the hydroxyl-functional thioether as a reactive diluent in thermally curable compositions.

13. A multilayer coating comprising at least two coating layers, wherein at least one of the layers is formed from the thermally curable composition according to claim 8.

14. A multilayer coated substrate, wherein the substrate is coated with a multilayer coating according to claim 13.

15. A method of producing the multilayer coating on a substrate, the method comprising the steps of:
(i) applying at least one basecoat composition on a substrate to form a basecoat layer and subsequently,
(ii) applying at least one topcoat composition, onto the basecoat composition to form a topcoat layer, followed by
(iii) curing of the fully cured coating layers at a temperature in a range from 20° C. to 200° C.,
wherein at least one basecoat composition or at least one topcoat composition is the thermally curable composition according to claim 8.
